(19) United States
(12) Patent Application Publication
(10) Pub. No.: US 2016/0120765 A1
(43) Pub. Date: May 5, 2016

(54) Zirconia Dental Articles and Methods Thereof

(71) Applicant: Sagemax Bioceramics, Inc., Federal Way, WA (US)

(72) Inventors: Sheng Dang, Federal Way, WA (US); Hua Dang, Federal Way, WA (US)

(73) Assignee: Sagemax Bioceramics, Inc.

(21) Appl. No.: 14/927,338

(22) Filed: Oct. 29, 2015

Related U.S. Application Data
(60) Provisional application No. 62/072,556, filed on Oct. 30, 2014.

(51) Int. Cl. A61K 6/02; B32B 18/00

(52) U.S. Cl. CPC A61K 6/025; B32B 18/00; C04B 2237/348; B32B 2255/26; B32B 2535/00

(57) ABSTRACT

Described herein are techniques for making a multiplayer zirconia sintered body. The techniques sinter a dental article to form the zirconia sintered body. The dental article includes multiple layers containing dental ceramic materials such as, zirconia oxide particles, yttria, a pigment, etc. In some implementations, the dental article may be partially sintered and coated using an organic material.

### CROSS REFERENCE TO RELATED PATENT APPLICATIONS

This application claims priority to U.S. Provisional Patent Application No. 62/072,556, filed on Oct. 30, 2014, entitled “Multilayer Zirconia Body and Methods,” which is hereby incorporated by reference in its entirety.

### BACKGROUND

Zirconia is well-studied ceramic materials for uses ranging from energy devices to environmentally-friendly goods. In clinical dentistry, zirconia ceramics have been used as crown materials in dental fixed restoration. Its durability, biocompatibility, natural esthetics and low cost when compared to alternative restorative materials make it the ideal solution for a variety of clinical applications. However, under conventional techniques, zirconia ceramics are made to have single and low light-transmission, which make difficult to mimic the appearance of natural teeth.

### SUMMARY

Embodiments of the present disclosure relate to methods for making a zirconia sintered body. The methods include sintering a dental article to form the zirconia sintered body. The dental article may include a first layer containing zirconia oxide particles and yttria in an amount of a first weight percentage, a second layer containing the zirconia oxide particles and yttria in an amount of a second weight percentage, and a third layer containing the zirconia oxide particles and yttria in an amount of a third weight percentage.

In some embodiment, the first weight percentage may be less than the second weight percentage that may be less than the third weight percentage. In some embodiments, at least one of the first layer, the second layer, or the third layer containing a pigment in an amount of a certain weight percentage. In these instances, a density of at least one of the first layer, the second layer, or the third layer of the zirconia sintered body less than 6.10 g/cm³ (e.g., 6.09 g/cm³). In some embodiments, the dental article may include a fourth layer containing the zirconia and yttria in an amount of a fourth weight percentage. In these instances, the fourth layer is above the third layer, and the fourth weight percentage is less than the third weight percentage.

Embodiments of the present disclosure relate to dental article for producing a dental prosthesis. The dental article may include a first layer containing zirconia oxide particles and yttria in an amount of a first weight percentage, a second layer containing the zirconia oxide particles and yttria in an amount of a second weight percentage, a third layer containing the zirconia oxide particles and yttria in an amount of a third weight percentage. In these instances, the second weight percentage is greater or less than the first weight percentage and the third weight percentage. The first layer is located below the second layer and the third layer, and the second layer is located between the first layer and the third layer. At least one of the first layer, the second layer, and the third layer includes a pigment in an amount of a certain weight percentage.

Embodiments of the present disclosure relate to methods for making a dental article related to a dental prosthesis. The methods may include providing a first mixture containing zirconia oxide particles and yttria in an amount of a first weight percentage, forming a first layer of the dental article by pressing the first mixture, providing a second mixture containing zirconia oxide particles and yttria in an amount of a second weight percentage, forming, above the first layer, a second layer of the dental article by pressing the second mixture, providing a third mixture containing zirconia oxide particles and yttria in an amount of a third weight percentage, and forming, above the second layer, a third layer of the dental article by pressing the third mixture to form a formed structure containing the first layer, the second layer, and the third layer. In some embodiments, the second weight percentage is greater or less than the first weight percentage and the third weight percentage. In these instances, at least one of the first layer, the second layer, and the third layer contains a pigment in an amount of a certain weight percentage.

The implementations also relate to dental articles for producing dental prosthesis and to methods for producing a dental article. A dental article may include a partially sintered ceramic article and a coating extending across at least a portion of a surface of the partially sintered ceramic article. A method may include providing a partially sintered ceramic article, and depositing a coating on at least a portion of a surface of the partially sintered ceramic article to form the dental article. For example, the coating may include an organic material.

In some implementations, the partially sintered ceramic article may include zirconia, and/or the coating does not include zirconia. In some implementations, the dental article is an injection-molded article. For example, the organic material may be injection-molded at a predetermined molding temperature using a mold on the surface of the partially sintered ceramic article.

In some implementations, the partially sintered ceramic article may include multiple layers, and the coating may extend across at least of a portion of an individual layer of the multiple layers.

In some implementations, the coating may include a plastic material or a water resistant/waterproof material. For example, the coating may include at least one of polyarylene sulfide, polyphenylene sulfide, acrylonitrile-butadiene-styrene, polyethylene, polymethyl methacrylate, polypropylene, polystyrene, polyvinyl chloride or polyethylene terephthalate.

The implementations further relate to methods for milling a sintered zirconia dental article. A method may include providing a sintered dental article. For example, the sintered dental article having multiple layers. The method may further include milling the sintered dental article using a milling technique. In these instances, the milling technique includes at least one of electronic discharge milling, water jet milling, laser milling, or controlled vaporization milling.

In some implementations, the sintered dental article formed at a temperature greater than 1000° C.

In some implementations, the multiple layers include a first layer, a second layer, and a third layer, and wherein a density of at least one of the first layer, the second layer, or the third layer of the zirconia sintered body less than 6.10 g/cm³ (e.g., 6.09 g/cm³).

In some implementation, the sintered dental article include one or more pigments.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The detailed description is described with reference to the accompanying figures.

FIG. 1 is a diagram illustrating an exemplary dental article.

FIG. 2 shows exemplary tables including various parameters of a multiplayer dental article.

FIG. 3 shows exemplary tables including various parameters of another multiplayer dental article.

FIG. 4 is a schematic diagram of an illustrative dental article for producing a dental prosthesis.

FIG. 5 is a diagram of an illustrative scheme for producing a dental article.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

“Substantially” or “essentially” means nearly totally or completely, for instance, 95%, 96%, 97%, 98%, 99% or greater of some given quantity.

Also herein, the recitations of numerical ranges by endpoints include all numbers subsumed within that range (e.g., at least 50 percent includes 50, 50.5, 55, 60, 67.5, 70, 73.8, etc.).

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “body” or variations thereof refers to a three-dimensional structure.

Dental ceramic materials have their unique patterns of response to heat when treated with high temperature for strengthening. Under conventional techniques, each layer of a multilayer dental article generally has the same chemical composition to have the same co-efficiency of thermal expansion (CTE). However, if chemical compositions of multiple layers are different, each layer may have a different CTE, sintering temperature, or sintering speed from other layers. This may causes the final restoration difficult to mimic the appearance of natural teeth. Embodiments of the present disclosure include a dental article having multiple layers that contain various amounts of dental ceramic materials (e.g., zirconia oxide particles, yttria, pigments, etc.). Using multiplayer techniques and appropriate combination of these dental ceramic materials, embodiments of the present disclosure produce a zirconia sintered body that mimic the appearance of natural teeth.

Under conventional techniques, flexural strength of a two-layer zirconia article including pigments is general less than 800 MPa, which appears noticeably lower than that of single-colored references being above 1000 MPa. This may be caused by granule packing defects and local enrichment of the pigments. Embodiments of the present disclosure also relate to a surprising discovery that a multilayer dental article containing one or more certain pigments (e.g., Ferric chloride) has a flexural strength of more than 1100 MPa (e.g., 1200˜1300).

Embodiments of the present disclosure relate to methods for making a zirconia sintered body. In some embodiments, the methods may include sintering a dental article to form the zirconia sintered body. In some embodiments, the dental article may include multiple layers such as, a first layer, a second layer, and a third layer. In these instances, the first layer may include zirconia oxide particles and yttria in an amount of a first weight percentage; the second layer may include the zirconia oxide particles and yttria in an amount of a second weight percentage; and the third layer may include the zirconia oxide particles and yttria in an amount of a third weight percentage. The first layer may be located below the second layer that is located below the third layer, and. In some embodiments, the first, second, and weight percentages may be different or substantially the same. For example, the first weight percentage is less than the second weight percentage that is less than the third weight percentage.

As used herein, the term “zirconia” refers to various stoichiometries for zirconium oxides, most typically ZrO₂, and may also be known as zirconium oxide or zirconium dioxide. For example, the zirconia may contain to 5% weight percent of oxides of one or more other chemical elements such as, for example, Y₂O₃, MgAl₂O₄, Al₂O₃, SiO₂, TiO₂, B₂O₃, Na₂O₃, etc.

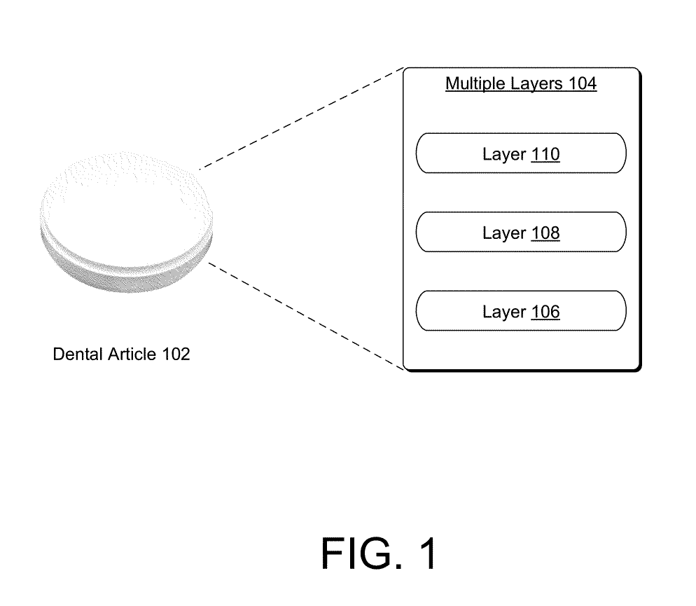

FIG. 1 is a diagram illustrating an exemplary dental article 102. The dental article 102 may be a dental blank, which includes a three-dimensional granular structure of various shapes of an article containing zirconia (e.g., disc or block or any shapes that can be fixedly attached to a dental milling machine). In some embodiments, the three-dimensional granular structure is not sintered. In these instances, the three dimensional In other embodiments, the three-dimensional granular structure may be partially sintered, pre-sintered, or soft sintered at, for example, a temperature of 900-1100° C. In some implementations, the terms “pre-sintering,” “partially sintered” and “soft sintered” interchangeably here to mean a reduction of size and/or number or the elimination of inter-particle pores in a granular structure by heating without melting the particles of the granular structure. Pre-sintering may be carried out at a temperature of, for example, about 900-1100° C. to, for example, facilitate the machine milling of molded zirconia disc/blank.

In some embodiments, pre-sintering or soft sintering may be performed on the cast or molded zirconia to obtain a green body with sufficient strength to be milled. After the dental article 102 of the specific dental restoration or green dental prosthesis is ready from the milling, sintering may be performed at a temperature of, for example about 1200-1600° C. to form a zirconia sintered body. In some embodiments, sintering may be performed on the milled and/or colored green dental prosthesis to obtain a dental prosthesis with final strength and optical characteristics such as, translucency and/or color intensity/chroma. The translucency may be measured using a percent light transmittance of at least 50% for wavelengths 350-700 nm through a 1 mm thickness of a ceramic body. For example, translucency of a body (e.g., the dental article 102 or the layer 106) may be represented using a total light transmittance percentage.

In some embodiments, the dental article 102 may be sintered at a temperature (e.g., around 1300-1600° C.). As used herein, the terms “sintering” and “primary sintering” and “final sintering” are used interchangeably. For example, before milling, primary/final sintering may be performed on the dental article 102 at a much higher temperature (around 1300-1600° C.) than pre-sintering. As used herein “sintered dental article” refers to a solid piece of material from which a dental prosthesis, such as a crown or bridge, may be formed by a subtractive milling.

After primary sintering, a sintered dental article may be obtain and ready for milling operations. As used herein, “milling” refers to abrading, polishing, controlled vaporization, electronic discharge milling (EDM), cutting by water jet or laser or any other method of cutting, removing, shaping or carving material. The milling is generally conducted predominantly by a machine. Sintered dental articles may be made in any desired shape or size, including cylinders, bars, cubes, polyhedra, ovoids, and plates.

The dental article 102 may include multiple layers 104, which may include various layers such as, a layer 106, a layer 108, and a layer 110. In some embodiments, a layer of the multiple layers 104 may correspond to a cervical area of a dental prosthesis or correspond to an incisal area of the dental prosthesis. For example, the layer 106 may correspond to a cervical area of the dental prosthesis; and the layer 110 may correspond to an incisal area of the dental prosthesis.

In some embodiments, the layer 106 may contains zirconia oxide particles and yttria in an amount of a first weight percentage; the layer 108 may contains zirconia oxide particles and yttria in an amount of a second weight percentage; and the layer 110 may contains zirconia oxide particles and yttria in an amount of a third weight percentage. In these instances, the first weight percentage may be less than the second weight percentage that may be less than the third weight percentage.

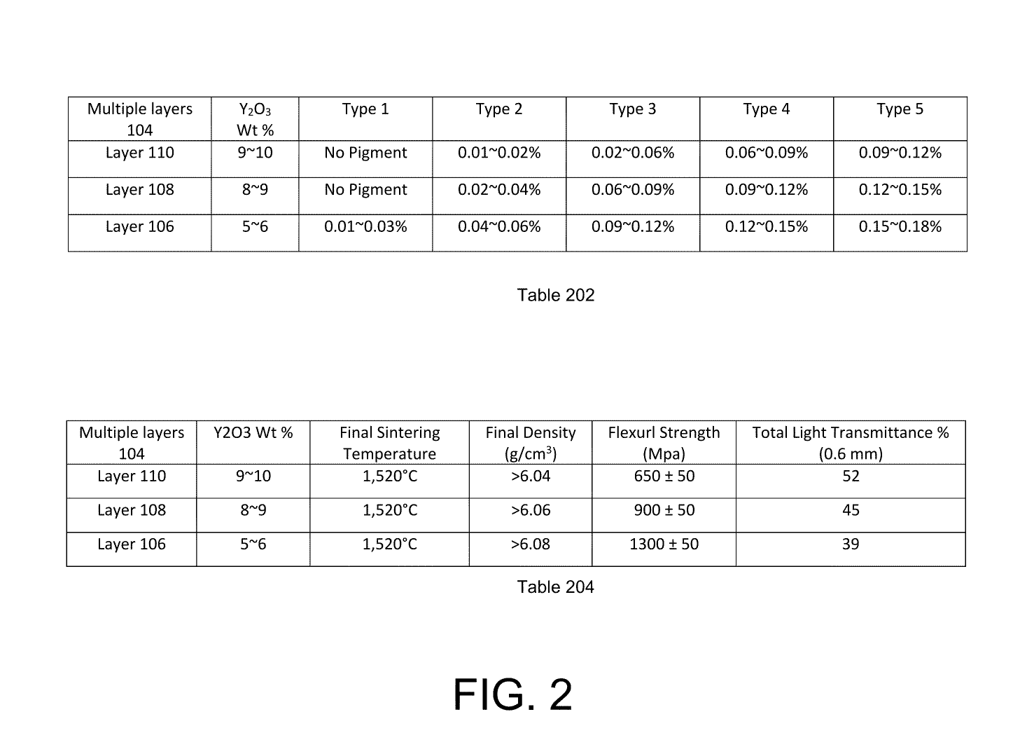

In some embodiments, the first weight percentage, the second weight percentage, and the third weight percentage may be from about 4.0 to about 11.0. For example, as illustrated in FIG. 2, the first weight percentage may be from about 5.0 to about 6.0. The second weight percentage may be from about 8.0 and about 9.0. The third weight percentage may be from about 9.0 and about 10.0. Various parameters of the layer 106, the layer 108, and the layer 110 may be found in a table 202 and a table 204.

In some embodiments, at least one of the layer 106, the layer 108, and the layer 110 may contain a pigment in an amount of a certain weight percentage. In these instances, the certain weight percentage the pigment may be from about 0.01 to about 0.2 weight percentage. For example, the certain weight percentage of the pigment in the first layer may be from about 0.01 to about 0.03. The certain weight percentage of the pigment in the second layer may be from about 0.02 to about 0.04. The certain weight percentage of the pigment in the third layer may be from about 0.01 to about 0.02. In some embodiments, the certain weight percentage of the pigment in the first layer, the certain weight percentage of the pigment in the second layer, and the certain weight percentage of the pigment in the third layer may be different, respectively. The pigment may include one or more of Ferric chloride, talc, titanium dioxide, Zinc oxide and iron oxides. In some embodiments, the pigment may be mixed with zirconia oxide particles and/or yttria in an individual layer of the dental article 102 in a form of ions (e.g., ions of Fe, Co, Nd, Ce, Mn, Cr). For example, FeCl₃ particles (e.g., powder) may be mixed with zirconia oxide particles to form the dental article 102, and may form fine oxide particles (e.g., Fe₂O₃) in a zirconia sintered body after the dental article 102 is sintered.

In some embodiments, a density of at least one of the first layer, the second layer, or the third layer of the zirconia sintered body less than 6.10 g/cm³ (e.g., 6.09 g/cm³). For example, the density of the first layer of the zirconia sintered body may be less than 4.0 g/cm³. The density of at least one of the third layer or the second layer of the zirconia sintered body is about 5.0 g/cm3 to about 7.0 g/cm³. As defined herein, a density of an article refers to mass of the article per unit volume. For example, a density of the dental article 102 or the layer 106 means mass of the dental article 102 or the layer 106 per unit volume.

In some embodiments, total light transactions of the layer 106, the layer 108, and the layer 110 are different. For example, a total light transaction of the layer 110 is 10% more than a total light transmittance percentage of the layer 108; and the total light transmittance percentage of the layer 108 is 10% more than a total light transaction of the layer 106.

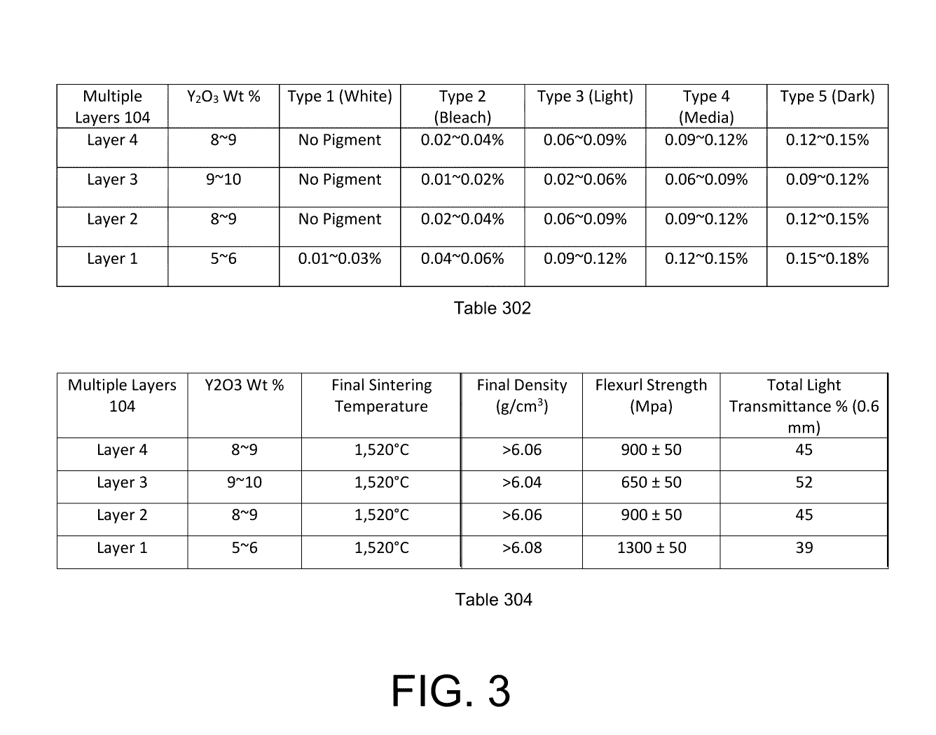

In some embodiments, the multiple layers 104 may include a fourth layer containing the zirconia and yttria in an amount of a fourth weight percentage. In these instances, the fourth layer may be above the third layer, and the fourth weight percentage may be less than the third weight percentage. For example, as illustrated in FIG. 3, various parameters of the multiple layers 104 including a fourth layer may be obtained in a table 302 and a table 304.

Embodiments of the present disclosure also relate to dental articles for producing dental prostheses. A dental article may include a first layer containing zirconia oxide particles and yttria in an amount of a first weight percentage, a second layer containing the zirconia oxide particles and yttria in an amount of a second weight percentage, and a third layer containing the zirconia oxide particles and yttria in an amount of a third weight percentage. In these instances, the first layer is located below the second layer and the third layer and the second weight percentage may be greater or less than the first weight percentage and the third weight percentage.

Embodiments of the present disclosure also relate to methods for making a dental article for producing dental prostheses. A method may include providing a first mixture including zirconia oxide particles and yttria in an amount of a first weight percentage. A first layer of the article may be formed by pressing the first mixture. The method may further include providing a second mixture including zirconia oxide particles and yttria in an amount of a second weight percentage. A second layer of the dental article may be formed above the first layer by pressing the second mixture. Further, the method may providing a third mixture including zirconia oxide particles and yttria in an amount of a third weight percentage. A third layer of the dental article may be formed by pressing the third mixture to form a formed structure including the first layer, the second layer, and the third layer.

A dental article of the dental articles may include multiple a first layer containing zirconia oxide particles and a first amount of yttria and a second layer containing the zirconia oxide particles and a second amount of yttria. The dental article may also include a third layer containing the zirconia oxide particles and a third amount of yttria. The first layer may be below the second layer and the third layer, and the second layer may be located between the first layer and the third layer. The second amount of yttria is greater or less than the first amount of yttria and the third amount of yttria. In some embodiments, the first layer is located below the third layer. In these instances, the second weight percentage may be greater or less than the first weight percentage and the third weight percentage. In some embodiments, at least one of the first layer, the second layer, and the third layer containing a pigment in an amount of a certain weight percentage.

In some embodiments, the first weight percentage, the second weight percentage, and the third weight percentage are from about 4.0 to about 11.0. In some embodiments, the certain weight percentage the pigment is from about 0.01 to about 0.2 weight percentage. In these instances, the certain weight percentage of the pigment in the first layer, the certain weight percentage of the pigment in the second layer, and the certain weight percentage of the pigment in the third layer are different, respectively, or substantially the same.

The implementations also relate to dental articles for producing dental prosthesis. For example, the dental prosthesis may include at least one of restoratives, replacements, inlays, onlays, veneers, full and partial crowns, bridges, implants, implant abutments, copings, anterior fillings, posterior fillings, cavity liners, or bridge frameworks.

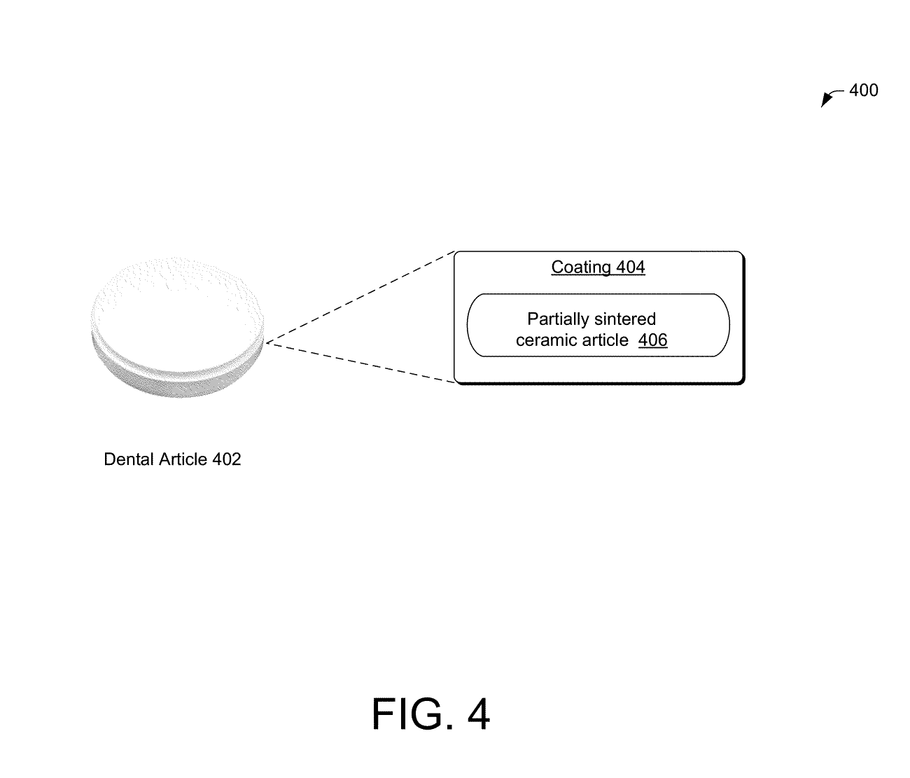

FIG. 4 is a schematic diagram 400 of an illustrative dental article for producing a dental prosthesis. The schematic diagram 400 includes a dental article 402, which may include a coating 404 and/or a partially sintered ceramic article 406. The dental article 402 and/or the partially sintered article 406 may be prepared in a variety of shapes and sizes to be used for producing dental prosthesis. For example, the shapes of the dental article 402 and/or the partially sintered ceramic article 406 may include square, circular, rectangular, cylindrical and triangular shapes.

The coating 404 may extend across at least a portion of the surface of the partially sintered ceramic article 406. For example, the coating 404 may extend across the surface of partially sintered ceramic article 406 such that sustainably all the surface of the partially sintered ceramic article 406 is covered by the coating 404. In this instance, the moisture in the air may be substantially prevented from the partially sintered ceramic article 406 such that the partially sintered ceramic article 406 may be in a dry condition for a certain time period.

In some implementations, moisture permeability of the partially sintered ceramic article 406 may be reduced by using the coating 404. For example, the partially sintered ceramic article 406 may be stored longer than those without using the coating 404.

In some implementations, the partially sintered ceramic article 406 may include a material that is not sintered to full density. For example, partially sintered ceramic materials are refractory, not reactive, and essentially inert during subsequent heating steps. The partially sintered ceramic article 406 may include various forms of silica, leucite, various forms of zirconia, alumina, magnesia, and/or aluminosilicate.

In some implementations, the partially sintered ceramic article 406 may be obtained by partially sintering the dental article 102, which include multiple layers. Accordingly, the partially sintered ceramic article 406 may further include multiple layers each including zirconia. In these instances, the coating 404 may extend across at least of a portion of each layer of the multiple layers. For example, the coating 404 may extend across the upper surface of layer 110, the bottom surface of layer 106, and the side surface of layers 110, 108, as well as 106.

The partially sintered ceramic article 406 may include zirconia, while in some implementations, the coating 404 does not include zirconia. The coating 404 may include an organic material such as a plastic material, a waterproof material, and/or a water resistant material. For example, the coating 404 may include at least one of polyarylene sulfide, polyphenylene sulfide, acrylonitrile-butadiene-styrene, polyethylene, polymethyl methacrylate, polypropylene, polystyrene, polyvinyl chloride or polyethylene terephthalate.

In some implementations, the dental article 402 is an injection-molded article. For example, an organic material may be injection-molded at a predetermined molding temperature using a mold on a surface of the partially sintered ceramic article 406 to form the coating 404.

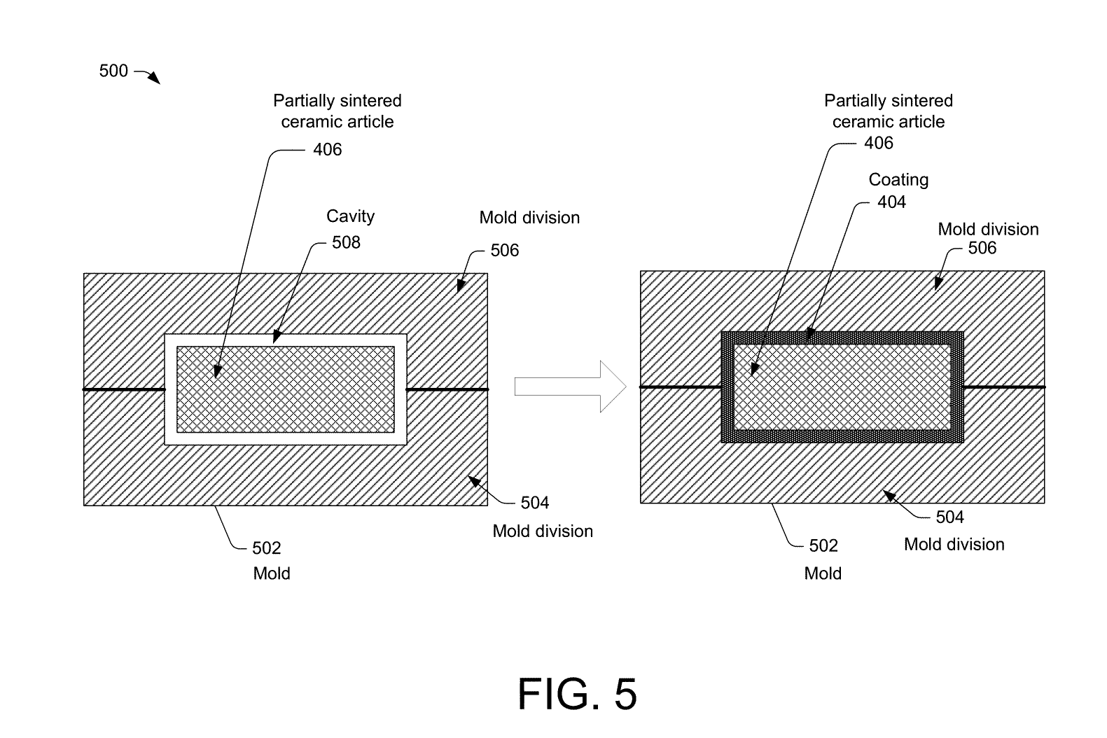

FIG. 5 is a diagram of an illustrative scheme 500 for producing the dental article 402. The scheme 500 may include a mold 502 for producing the dental article 402. The mold 502 may include a mold division 504 and a mold division 506. The partially sintered ceramic article 406 may be placed inside of the mold 502. In these instances, there may be a cavity 508 located between the partially sintered ceramic article 406 and the mold divisions 504 as well as 506.

In some implementations, an organic material, such as a plastic material in a molten state, may be injected into the mold 502 such that the cavity 508 is filled with the organic material to form the coating 404 extending the surface of the partially sintered ceramic article 406. After the coating 404 cools down, the dental article 402 may be obtained accordingly.

The implementations further relate to methods for milling a sintered zirconia dental article. A method may include providing a sintered dental article. For example, the sintered dental article having multiple layers. The method may further include milling the sintered dental article using a milling technique. In these instances, the milling technique includes at least one of electronic discharge milling, water jet milling, laser milling, or controlled vaporization milling.

In some implementations, the sintered dental article formed at a temperature greater than 1000° C. For example, the sintered dental article is produced by sintering the dental article of claim 15 at a temperature from about 1,300 to about 1,600° C.

In some implementations, the sintered dental article is produced using any method of claims 1-14. For example, the multiple layers include a first layer, a second layer, and a third layer, and wherein a density of at least one of the first layer, the second layer, or the third layer of the zirconia sintered body less than 6.10 g/cm³(e.g., 6.09 g/cm³). In some implementation, the sintered dental article includes one or more pigments.

### Example Embodiments

1. A method for making a zirconia sintered body, the method comprising: sintering a dental article to form the zirconia sintered body, the dental article comprising: a first layer comprising zirconia oxide particles and yttria in an amount of a first weight percentage, a second layer comprising the zirconia oxide particles and yttria in an amount of a second weight percentage, and a third layer comprising the zirconia oxide particles and yttria in an amount of a third weight percentage, the first weight percentage less than the second weight percentage that is less than the third weight percentage, at least one of the first layer, the second layer, or the third layer comprising a pigment in an amount of a certain weight percentage, a density of at least one of the first layer, the second layer, or the third layer of the zirconia sintered body less than 6.09 g/cm³.

2. The method of embodiment 1, wherein the first weight percentage, the second weight percentage, and the third weight percentage are from about 4.0 to about 11.0.

3. The method of claim 1, wherein the first weight percentage is from about 5.0 to about 6.0.

4. The method of embodiment 1, wherein the second weight percentage is from about 8.0 and about 9.0.

5. The method of embodiment 1, wherein the third weight percentage is from about 9.0 and about 10.0.

6. The method of embodiment 1, wherein the certain weight percentage the pigment is from about 0.01 to about 0.2 weight percentage.

7. The method of embodiment 1, wherein the certain weight percentage of the pigment in the first layer, the certain weight percentage of the pigment in the second layer, and the certain weight percentage of the pigment in the third layer are different, respectively.

8. The method of embodiment 1, wherein the certain weight percentage of the pigment in the first layer is from about 0.01 to about 0.03.

9. The method of embodiment 1, wherein the certain weight percentage of the pigment in the second layer is from about 0.02 to about 0.04.

10. The method of embodiment 1, wherein the certain weight percentage of the pigment in the third layer is from about 0.01 to about 0.02.

11. The method of embodiment 1, wherein the density of the first layer of the zirconia sintered body is less than 4.0 g/cm³.

12. The method of embodiment 1, wherein the density of at least one of the third layer or the second layer of the zirconia sintered body is about 5.0 g/cm³ to about 7.0 g/cm³.

13. The method of embodiment 1, wherein the dental article comprises: a fourth layer comprising the zirconia and yttria in an amount of a fourth weight percentage, wherein the fourth layer is above the third layer, and wherein the fourth weight percentage is less than the third weight percentage.

14. The method of embodiment 1, wherein a total light transaction of the third layer is 10% more than a total light transmittance of the first layer.

15. A dental article for producing a dental prosthesis, the dental article comprising: a first layer comprising zirconia oxide particles and yttria in an amount of a first weight percentage; a second layer comprising the zirconia oxide particles and yttria in an amount of a second weight percentage; and a third layer comprising the zirconia oxide particles and yttria in an amount of a third weight percentage, wherein: the second weight percentage is greater or less than the first weight percentage and the third weight percentage, the first layer is located below the second layer and the third layer, the second layer is located between the first layer and the third layer, and at least one of the first layer, the second layer, and the third layer comprises a pigment in an amount of a certain weight percentage.

16. A method for making a dental article related to a dental prosthesis, the method comprising: providing a first mixture comprising zirconia oxide particles and yttria in an amount of a first weight percentage; forming a first layer of the dental article by pressing the first mixture; providing a second mixture comprising zirconia oxide particles and yttria in an amount of a second weight percentage; forming, above the first layer, a second layer of the dental article by pressing the second mixture; providing a third mixture comprising zirconia oxide particles and yttria in an amount of a third weight percentage; and forming, above the second layer, a third layer of the dental article by pressing the third mixture to form a formed structure comprising the first layer, the second layer, and the third layer, the second weight percentage being greater or less than the first weight percentage and the third weight percentage, at least one of the first layer, the second layer, and the third layer comprising a pigment in an amount of a certain weight percentage.

17. The dental article or the method of embodiments 15 or 16, wherein the first weight percentage, the second weight percentage, and the third weight percentage are from about 4.0 to about 11.0.

18. The dental article or the method of embodiments 15 or 16, wherein the certain weight percentage the pigment is from about 0.01 to about 0.2 weight percentage.

19. The dental article or the method of embodiments 15 or 16, wherein the certain weight percentage of the pigment in the first layer, the certain weight percentage of the pigment in the second layer, and the certain weight percentage of the pigment in the third layer are different, respectively.

20. A dental article for producing a dental prosthesis, the dental article comprising: a partially sintered ceramic article; and a coating extending across at least a portion of a surface of the partially sintered ceramic article, the coating having an organic material.

21. A method for producing a dental article, the method comprising: providing a partially sintered ceramic article; and depositing a coating on at least a portion of a surface of the partially sintered ceramic article to form the dental article, the coating having an organic material.

22. The dental article or the method of embodiments 20 and 21, wherein the partially sintered ceramic article comprise zirconia.

23. The dental article or the method of embodiments 20 and 21, wherein the dental article is an injection-molded article, and wherein the organic material is injection-molded at a predetermined molding temperature using a mold on a surface of the partially sintered ceramic article.

24. The dental article or the method of embodiments 20 and 21, wherein the partially sintered ceramic article comprises a plurality of layers, and wherein the coating extends across at least of a portion of an individual layer of the plurality of layers.

25. The dental article or the method of embodiments, 20 and 21, wherein the partially sintered ceramic article is obtained from the dental article of claim 15.

26. The dental article or the method of embodiments 20 and 21, wherein the coating does not comprise zirconia.

27. The dental article or the method of embodiments 20 and 21, wherein the coating comprises a plastic material. 28. The dental article or the method of embodiments 20 and 21, wherein the coating comprises a waterproof material.

29. The dental article or the method of embodiments 20 and 21, wherein the coating comprises at least one of polyarylene sulfide, polyphenylene sulfide, acrylonitrile-butadiene-styrene, polyethylene, polymethyl methacrylate, polypropylene, polystyrene, polyvinyl chloride or polyethylene terephthalate.

30. A method for milling a sintered zirconia dental article, the method comprising: providing a sintered dental article, the sintered dental article having multiple layers; and milling the sintered dental article using a milling technique.

31. The method of embodiment 30, wherein the milling technique includes at least one of electronic discharge milling, water jet milling, laser milling, or controlled vaporization milling.

32. The method of embodiment 31, wherein the sintered dental article formed at a temperature greater than 1000° C.

33. The method of embodiment 31, wherein the multiple layers include a first layer, a second layer, and a third layer, and wherein a density of at least one of the first layer, the second layer, or the third layer of the zirconia sintered body less than 6.09 g/cm³.

34. The method of embodiment 31, wherein the sintered dental article includes one or more pigments.

35. The method of embodiment 31, wherein the sintered dental article is produced using any method of claims 1-14.

36. The method of embodiment 31, wherein the sintered dental article is produced by sintering the dental article of claim 15 at a temperature from about 1,300 to about 1,600° C.

What is claimed is:

1. A dental article for producing a dental prosthesis, the dental article comprising:
a first layer comprising yttria in an amount of a first weight percentage and zirconia oxide particles;
a second layer comprising yttria in an amount of a second weight percentage and the zirconia oxide particles; and
a third layer comprising yttria in an amount of a third weight percentage and the zirconia oxide particles, wherein:
the first layer is located below the second layer and the third layer,
the second layer is located between the first layer and the third layer, and
at least one of the first layer, the second layer, and the third layer comprises a pigment in an amount of a certain weight percentage.

2. The dental article of claim 1, wherein the second weight percentage is greater or less than the first weight percentage and the third weight percentage, and wherein the first weight percentage, the second weight percentage, and the third weight percentage are from about 4.0 to about 11.0.

3. The dental article of claim 1, wherein the first weight percentage is from about 5.0 to about 6.0, the second weight percentage is from about 8.0 and about 9.0, and the third weight percentage is from about 9.0 and about 10.0.

4. The dental article of claim 3, wherein the certain weight percentage of the pigment in the first layer is from about 0.01 to about 0.03, wherein the certain weight percentage of the pigment in the second layer is from about 0.02 to about 0.04, and wherein the certain weight percentage of the pigment in the third layer is from about 0.01 to about 0.02.

5. The dental article of claim 1, wherein, wherein the certain weight percentage the pigment is from about 0.01 to about 0.2 weight percentage, and wherein the certain weight percentage of the pigment in the first layer, the certain weight percentage of the pigment in the second layer, and the certain weight percentage of the pigment in the third layer are different, respectively.

6. The dental article of claim 1, wherein a density of the first layer of the dental article is less than 4.0 g/cm³, and a density of at least one of the third layer or the second layer of the dental article is about 5.0 g/cm³ to about 7.0 g/cm³.

7. The dental article of claim 1, further comprising:
a fourth layer comprising the zirconia and yttria in an amount of a fourth weight percentage, wherein the fourth layer is above the third layer, and wherein the fourth weight percentage is less than the third weight percentage.

8. The dental article of claim 1, wherein a total light transaction of the third layer is 10% more than a total light transmittance of the first layer.

9. The dental article of claim 1, further comprising:
a coating extending across at least a portion of a surface of the dental article, the coating having an organic material, the dental article partially sintered.

10. The dental article of claim 9, wherein the dental article is an injection-molded article, and wherein the organic material is injection-molded at a predetermined molding temperature using a mold on a surface of the partially sintered dental article.

11. The dental article of claim 9, wherein the coating does not comprise zirconia, and the coating comprises a waterproof material.

12. The dental article of claim 9, wherein the coating comprises at least one of polyarylene sulfide, polyphenylene sulfide, acrylonitrile-butadiene-styrene, polyethylene, polymethyl methacrylate, polypropylene, polystyrene, polyvinyl chloride or polyethylene terephthalate.

13. A method for making a zirconia sintered body, the method comprising:
sintering a dental article to form the zirconia sintered body, the dental article comprising:
a first layer comprising zirconia oxide particles and yttria in an amount of a first weight percentage,
a second layer comprising the zirconia oxide particles and yttria in an amount of a second weight percentage, and
a third layer comprising the zirconia oxide particles and yttria in an amount of a third weight percentage, at least one of the first layer, the second layer, or the third layer comprising a pigment in an amount of a certain weight percentage.

14. The method of claim 13, wherein a density of at least one of the first layer, the second layer, or the third layer of the zirconia sintered body less than 6.09 g/cm³, and a total light transaction of the third layer is 10% more than a total light transmittance of the first layer.

15. The method of claim 13, wherein the first weight percentage, the second weight percentage, and the third weight percentage are from about 4.0 to about 11.0.

16. The method of claim 13, wherein the first weight percentage is from about 5.0 to about 6.0, the second weight percentage is from about 8.0 and about 9.0, and the third weight percentage is from about 9.0 and about 10.0.

17. The method of claim 13, wherein the certain weight percentage the pigment is from about 0.01 to about 0.2 weight percentage, and the certain weight percentage of the pigment in the first layer, the certain weight percentage of the pigment in the second layer, and the certain weight percentage of the pigment in the third layer are different, respectively.

18. The method of claim 13, wherein the certain weight percentage of the pigment in the first layer is from about 0.01 to about 0.03, wherein the certain weight percentage of the pigment in the second layer is from about 0.02 to about 0.04, and wherein the certain weight percentage of the pigment in the third layer is from about 0.01 to about 0.02.

19. The method of claim 13, wherein a density of the first layer of the zirconia sintered body is less than 4.0 g/cm³, and a density of at least one of the third layer or the second layer of the zirconia sintered body is about 5.0 g/cm³ to about 7.0 g/cm³.

20. The method of claim 13, wherein the dental article comprises:
a fourth layer comprising the zirconia and yttria in an amount of a fourth weight percentage, wherein the fourth layer is above the third layer, and wherein the fourth weight percentage is less than the third weight percentage, wherein the first weight percentage is less than the second weight percentage that is less than the third weight percentage.
